(19) United States
(12) Patent Application Publication
(10) Pub. No.: US 2023/0173005 A1
(43) Pub. Date: Jun. 8, 2023

(54) USE OF BROWN FLAMMULINA VELUTIPES EXTRACT FOR INHIBITING ACUTE KIDNEY INJURY CAUSED BY CISPLATIN

(71) Applicant: ASIA UNIVERSITY, TAICHUNG CITY (TW)

(72) Inventors: MING-MING LEE, Taichung City (TW); GUAN-JHONG HUANG, Taichung City (TW)

(21) Appl. No.: 17/885,279

(22) Filed: Aug. 10, 2022

(30) Foreign Application Priority Data
Dec. 7, 2021 (TW) 110145713

(51) Int. Cl. A61K 36/07

(52) U.S. Cl. CPC A61K 36/07

(57) ABSTRACT

The present invention relates to the use of a brown Flammulina velutipes extract for inhibiting acute kidney injury caused by cisplatin. With the water extract of brown Flammulina velutipes, which reduces the renal histological changes, the production of creatinine, blood urea nitrogen in the acute kidney injury caused by cisplatin.

### CROSS-REFERENCE TO RELATED APPLICATION

This application claims the benefit of Taiwan Patent Application No. 110145713, filed on Dec. 7, 2021, in the Taiwan Intellectual Property Office, the disclosure of which is incorporated herein in its entirety by reference.

### BACKGROUND OF THE INVENTION

### 1. Field of the Invention

The present invention relates to a use of a brown Flammulina velutipes extract for inhibiting an acute kidney injury, and more particularly, to a use for inhibiting a cisplatin-induced acute kidney injury.

### 2. Description of the Related Art

The incidence of kidney disease in Taiwan still remains at a high level. Acute renal failure is one of common kidney diseases. A kidney structure of a patient may be damaged or loses functions in a short period of time due to various causes, and even worsen into kidney fibrosis, and the kidneys may eventually weaken to increase risk of death. Brown Flammulina velutipes is one of the world's major edible fungi, and it is spotlighted by many people because of the nutritional value and taste. Meanwhile, according to related studies, brown Flammulina velutipes has various pharmacological properties, and includes anti-tumor, anti-inflammatory and antioxidant properties. This signifies that brown Flammulina velutipes has very large bioactive potential.

According to statistics data of the Ministry of Health and Welfare of Taiwan in 2021, acute renal failure and chronic kidney disease have always been in the top 20 diseases in Taiwan's health insurance spending, from 2016 to 2020. Meanwhile, in 2006, the International Society of Nephrology (ISN) and the International Federation Kidney Foundations (IFKF) proposed designating the second Thursday of March every year as World Kidney Day. In other words, it can be seen that kidney disease also has a certain effect on the world population.

Meanwhile, cancer has always plagued many people. Although the use of chemical drugs for treating cancer has gradually developed into new possibilities, it is known that some chemotherapeutic drugs have various side effects. For example, cisplatin is a drug commonly used to treat head and neck cancer, ovarian cancer, and testicular cancer. However, mainly due to adverse effects on the kidneys, the patient's blood urea nitrogen (BUN) and creatinine are required to be monitored before treatment. Therefore, there is a limit to clinical application.

A nephrotoxic drug is one of causes to the formation of nephritic factor in the acute kidney injury. Currently, studies on animal models for simulating the acute kidney injury by injecting cisplatin have been gradually conducted. Increased oxidative stress, apoptosis and increased inflammation in the kidney are considered as potential mechanisms causing nephrotoxicity. After drug injection, cisplatin may be metabolically activated to reactive oxygen species (ROS) when passing through renal tubular epithelial cells from plasma, affect the antioxidant system of cells, and interact with different cellular components or macromolecules, thereby causing protein and lipid oxidative damage (malondialdehyde (MDA)). In addition, the oxidation reaction induced by ROS may activate signal proteins including NF-κB. In addition, ROS also causes damage to cellular DNA in the kidney, thereby causing caspase-3-related cell death. Meanwhile, when the damage is caused, various proinflammatory cytokines, such as IL-1β, IL-6 and TNF-α, are generated, and the production of iNOS and COX-2 serving as inflammatory substances are simultaneously induced.

The brown Flammulina velutipes (brown enoki mushroom) is a brown product of Flammulina velutipes gradually circulating in the market. According to the relevant documents, brown Flammulina velutipes has anti-inflammatory, antioxidant and renal-protective effects; and brown Flammulina velutipes has antioxidant and anti-inflammatory properties against L929 cells. However, the relationship between brown Flammulina velutipes and acute kidney injury has not been studied.

In conclusion, there are needs for further studies on whether brown Flammulina velutipes can be used to inhibit or slow the acute kidney injury from nephrotoxic drugs.

### SUMMARY OF THE INVENTION

In view of the above background, as a main direction for the research of the present invention, a mouse model of the acute renal failure induced by cisplatin has been derived based on the antioxidant and anti-inflammatory properties of brown Flammulina velutipes. In addition, according to in vivo tests and related experiments, renal cytoprotective functions of a water extract of brown Flammulina velutipes and Amipostine (cytoprotective agent) are compared. In addition, it was further investigated how the water extract of brown Flammulina velutipes controls signaling pathways that alleviate symptoms associated with the acute renal failure such as increased renal oxidative stress, and renal cell inflammation and apoptosis.

Brown Flammulina velutipes of the present invention refers to brown Flammulina velutipes grown at Wansheng Biotechnology Farm. Because a mushroom cap of brown Flammulina velutipes is remarkably dark in color, a mushroom stem appears white to light brown, and a mushroom cap appears dark brown (see FIG. 1). Brown Flammulina velutipes is different from enoki mushrooms sold in the market, and has the texture smoother and softer than the commercially available enoki mushroom when eaten.

The brown Flammulina velutipes strain was compared with the sequences of three enoki mushrooms published in the National Center for Biotechnology Information (NCBI) (see the following URL: https://www.ncbi.nlm.nih.gov/genome/browse#!/eukaryotes/16873/). Each similarity is 99.19% (see the following URL: http://enve-omics.ce.gatech.edu/ani/results?jid=611f0787945ab), 97.52% (see the following URL: http://enve-omics.ce.gatech.edu/ani/results?jid=611dcf669e202), and 96.90% (see the following URL: http://enve-omics.ce.gatech.edu/ani/results?jid=611f07c38af45). Accordingly, it was confirmed that the above enoki mushrooms also belong to Flammulina velutipes, and additionally have deposited in the incorporated foundation Food Industry Development Institute. The strain is WS-GNM-0910, the deposit date is Aug. 26, 2020, and the deposit number is BCRC930222.

Hereinafter, specific embodiments will be described in detail with reference to the accompanying drawings, for the further understanding of the object, technical content, features, and effects achieved thereby of the present invention.

### BRIEF DESCRIPTION OF THE DRAWINGS

FIG. 1 is a view schematically showing the appearance of brown Flammulina velutipes used in the present invention.

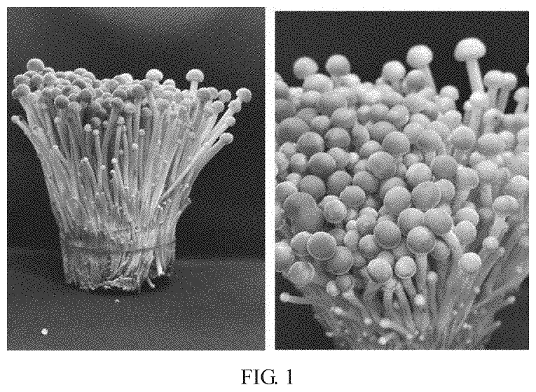

FIG. 2 is a schematic view showing the effect of the control group, the cisplatin induction group, the positive control group, and the low-middle-high dose brown Flammulina velutipes water extract group on a body weight of the mouse.

FIG. 3 is a schematic view showing the effect of the control group, the cisplatin induction group, the positive control group, and the low-middle-high dose brown Flammulina velutipes water extract group on a kidney function of the mouse.

FIG. 4 is a schematic view showing the effect of the control group, the cisplatin induction group, the positive control group, and the low-middle-high dose brown Flammulina velutipes water extract group on an NO free radicals in mouse serum.

FIG. 5 is a schematic view showing the effect of the control group, the cisplatin induction group, the positive control group, and the low-middle-high dose brown Flammulina velutipes water extract group on TNF-α, IL-1β and IL-6 of the mouse.

FIG. 6 is a schematic view showing the effect of the control group, the cisplatin induction group, the positive control group, and the low-middle-high dose brown Flammulina velutipes water extract group on iNOS and COX-2 of the mouse.

FIG. 7 is a schematic view showing the effect of the control group, the cisplatin induction group, the positive control group, and the low-middle-high dose brown Flammulina velutipes water extract group on TLR4/NFκB of the mouse.

FIG. 8 is a schematic view showing the effect of the control group, the cisplatin induction group, the positive control group, and the low-middle-high dose brown Flammulina velutipes water extract group on SOD, catalase and GPX1 of the mouse.

FIG. 9 is a schematic view showing the effect of the control group, the cisplatin induction group, the positive control group, and the low-middle-high dose brown Flammulina velutipes water extract group on the expression level of Nrf2/HO-1 of the mouse.

FIG. 10 is a schematic view showing the effect of the control group, the cisplatin induction group, the positive control group, and the low-middle-high dose brown Flammulina velutipes water extract group on Bcl-2 and Bax of the mouse.

FIG. 11 is a schematic view showing the effect of the control group, the cisplatin induction group, the positive control group, and the low-middle-high dose brown Flammulina velutipes water extract group on LC3B, p62 and Beclin-1 of the mouse.

FIG. 12 is a schematic view showing the effect of the control group, the cisplatin induction group, the positive control group, and the low-middle-high dose brown Flammulina velutipes water extract group on PI3K/AKT pathways of the mouse.

### DETAILED DESCRIPTION OF THE PREFERRED EMBODIMENTS

Hereinafter, embodiments of the present invention will be described in detail with reference to exemplary drawings. In addition to these detailed descriptions, the present invention may be broadly implemented in other embodiments, and simple substitutions, modifications and equivalent variations of the above embodiments are all included within the scope of the present invention, and subject to the following claims. Various specific details are provided in the description in the specification to more fully understand the present invention. However, the present invention is still embodied without certain details omitted partially or entirely. In the above details, the descriptions for known steps or elements are omitted to avoid that the present invention is unnecessarily limited. In the drawings, the same or similar components may be denoted by the same or similar reference numerals. In particular, the drawings are illustrative only, and may not represent the actual size or quantity of the components and some details may not be fully illustrated for the brevity of the drawings.

Treatment of Materials

First, 200 g of brown Flammulina velutipes was added to 1 L of secondary filtered water (as the extraction solvent), boiled at 100° C., the collected liquid was filtered and freeze-dried and brown Flammulina velutipes water extract was obtained and stored in a dry box.

Animal Model

Male ICR mice aged 7-8 weeks (each weighing about 30-32 grams) were divided into 6 groups, 10 mice per group, and the six groups correspond to a control group, an induction group, a positive control group and low-medium-high dose brown Flammulina velutipes water extract groups. The entire process was limited to 10 days. The mice were injected on day 7 to induce cisplatin and sacrificed on day 10. Both of the induction and the sacrifice were performed in a fasting state.

The control group is a blank control group, which was provided with only food without treatment, and treated with fasting on the day before sacrifice; The induction group is a cisplatin induction group, which was provided with food normally, 20 mg/Kg of cisplatin was injected intraperitoneally on day 7 of the process, and fasting was performed on the day before induction; The positive control group used Amifostine, was provided with food normally, and, on day 7, cisplatin was injected and then Amifostine 200 mg/Kg was intraperitoneally injected 30 minutes later.

The dosage range of brown Flammulina velutipes water extract (WFV) is 250 mg/kg to 1000 mg/kg, and 250 mg/Kg, 500 mg/Kg, and 1000 mg/Kg of brown Flammulina velutipes water extracts (WFV) were administered with pipettes in the order of low-medium-high doses, respectively. Prior to the administration, WFV was ground and diluted with 1% carboxymethyl cellulose (CMC), and a total volume of 0.1 mL of drug was administered each time. The administration was performed for 10 consecutive days while feeding at around 10 a.m. Normal food was provided during the above period. On day 7, the administration was performed in the morning, and then cisplatin induction of 20 mg/Kg was performed again. On the day before induction, all three groups were treated on an empty stomach.

Weight Measurement

For mice in each group, body weight was measured before administration from day 1 to the day before sacrifice (day 9), and changes in body weight were observed over 10 days.

Measurement on Kidney Weight/Body Weight Ratio

Kidneys were removed from the mouse body, and adrenal glands and surrounding fat were removed, washed with saline and then weighed immediately. The weights of the both kidneys were obtained and compared with the weight of the mouse measured on the last day, and the difference between the kidney/weight ratios of the mice was calculated.

Kidney Function Test

The mice were sacrificed to collect corresponding blood. After storage at room temperature for 30 minutes, the blood was centrifuged at 3500 rpm for 20 minutes in a centrifugal separator at 4° C. After the centrifugation of blood, the blood supernatant was collected and sent to a laboratory to measure contents of blood urea nitrogen (BUN) and creatinine.

Nitrite Analysis

Mouse serum was taken by 100 μL each and added to a new 96-well microassay plate. Then, Griess Reagent was added by the same amount of 50 μL. The Griess reagent was obtained by mixing an aqueous solution of N-(1-naphthyl)ethylenediamine with a 1% sulfanilamide solution dissolved in 5% phosphoric acid in the volumetric ratio of 1:1. Thereafter, the absorbance was measured at a wavelength of 540 nm using a microplate spectrometer (Molecular Devices), so that the ability of removing NO free radicals was measured in each group.

Malondialdehyde (MDA) Analysis

Kidney was added to 50 Nm of a sodium phosphate solution, and centrifuged for 5 minutes in an ice bath by using a homogenizer at 1400 rpm. Supernatant or different concentrations of MDA standard were taken by 400 μL and 0.2% of BHT was added by 12 μL, uniformly mixed with 0.4% of TBA color reagent by 400 μL, and then vibrated for 1 minute. The reacted solution was heated at 90° C. for 45 minutes, cooled in an ice bath, added with n-butanol extract in the ratio of 1:1, and centrifuged at 13000 rpm for 5 minutes. The supernatant is an MDA standard solution or a kidney homogenate MDA derivative, and an absorbance value at 535 nm was read and analyzed with a microplate spectrometer.

Cytokine Analysis

After sacrificing the mice to collect corresponding blood, and serum was separated and then diluted 10-fold or 5-fold. TNF-α, IL-6 and IL-1β were analyzed according to the multiple antibody sandwich principle by using BioLegend Company's ELISA kits.

In a 96-well microassay plate, 100 μL of capture antibody was added to each well, and placed at room temperature overnight. Then, 100 μL of standard solution and cell culture supernatant of unknown concentration were taken out, put in each well, reacted at room temperature for 2 hours, and then, washed 3 times with Wash buffer. Then, 100 μL of detection antibody was added and reacted at room temperature for 2 hours. Then, it was washed 3 times with Wash buffer, added with 100 μL of streptaviidine horseradish peroxidase (HRP), and blocked from light at room temperature for 30 minutes. Then, it was washed 3 times with Wash buffer, added with 100 μL of Tetramethylbenzidine (TMB) under the light blocking condition, and placed at room temperature for 20 minutes. Finally, 2 equivalents of 100 μL of sulfuric acid as a stop solution was added, and the change in absorbance at a wavelength of 450 nm was measured with a microplate spectrometer. The concentration of cytokines in serum was additionally calculated.

Western Blotting

The kidneys were homogenized with a PBS solution and diluted 10 folds to form a homogeneous solution, and the homogeneous solution was centrifuged at 12,000 rpm at 4° C. for 20 minutes. The supernatant was taken to quantify each protein concentration, and treated to have a uniform concentration. A protein was added to a protein dye to form a sample, placed in a drying bath at 100° C., and heated for 5 minutes. Then, the sample was injected into 10%, 15% or 8% of colloid prepared with 40% of acrylamide/bisN,N-methylenbisacrylamide, in which the ratio of acrylamide to bisN,N-methylenbisacrylamide was 29:1. First, the sample was collected while running the sample by a running buffer at 100 volts for 10 minutes. Thereafter, a voltage of 130 volts or 110 volts was selected according to the molecular weight of the protein, a separation of the colloid was started, and the colloid was taken out after about 60 to 80 minutes. Colloidal proteins were placed in a transfer buffer at a voltage of about 130 volts for 70 minutes and transferred into transfer membranes.

After completion of the transfer, blocking was performed for 1 hour using milk. Then, it was washed 3 times with TBST, a primary antibody was added after completion of washing, placed in an icebox and vibrated overnight. The primary antibody was recovered in the next morning, and washed 3 times with TBST, and a secondary antibody was added and placed in a shaker for 1 hour. Then, after washing with TBST 3 times, it was put in a low-illuminance color solution (Luminata Classico Western HRP Substract; ECL, Thermo, Scientific Hudson, USA) and immersed for about 2 minutes, and a PVDF film was developed using a photographic system (Kodak Molecular) and color development software (version 4.0.5, Eastman Kodak Company, Rochester, N.Y.). In addition, image data were acquired and analyzed by the Image J system.

The Sources of the Used Antibodies are as Follows.

Antibodies to iNOS, NF-κB, HO-1, Nrf2, p38 and BAX: purchased from Abcam (Cambridge, UK, USA);

Antibodies to COX-2, Catalase, SOD1, GPX3, and TLR4: purchased from Gene Tex (San Antonio, Tex., USA); and

Antibody to β-actin: purchased from Millipore (Billerica, Mass., USA).

Statistical Analysis

Values are expressed as mean±standard deviation (Mean±SD) for each group. Statistical analysis was conducted by multiple comparison analysis test, and experimental data were repeated at least 3 times. Student's T-test was adopted for the statistical method, and a p-value of less than 0.05 was considered significant data.

### Example 1

Effect of cisplatin on body weight and kidney weight of mouse and protective properties of brown Flammulina velutipes water extract

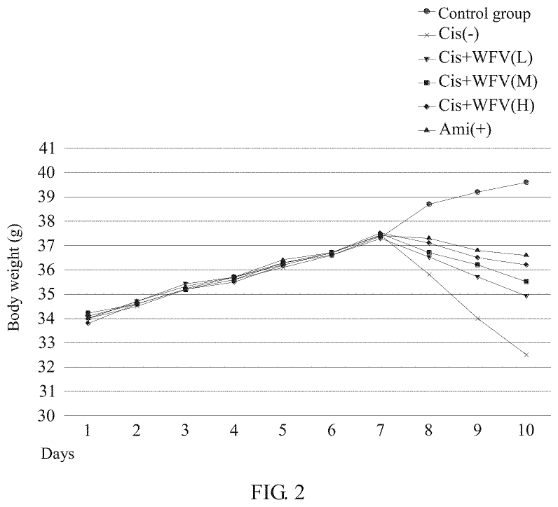

Referring to FIG. 2, FIG. 2 is a schematic view showing the effect of the control group, the cisplatin induction group, the positive control group, and the low-middle-high dose brown Flammulina velutipes water extract group on a body weight of the mouse. In FIG. 2, N is a control group, Cis(−) is a cisplatin induction group, Ami(+) is a positive control using Amifostine as nephroprotectant, and Cis+WFV(L), Cis+WFV(M), and Cis+WFV(H) are experimental groups of brown Flammulina velutipes water extract added to cisplatin in the order of low, medium, and high doses, respectively.

In the group of mice administered with cisplatin, a situation of weight loss was exhibited after induction. This signifies that the induction of cisplatin was successful. Meanwhile, in the group receiving brown Flammulina velutipes water extract, the situation of weight loss was decreased when the dose of brown Flammulina velutipes water extract is increased. This situation is most pronounced with 1000 mg/kg of brown Flammulina velutipes water extract (WFV).

Due to the acute kidney inflammation, the kidneys swell and weight is increased. In the ratio of kidney weight to body weight of the mouse, the ratio value of the cisplatin group was significantly higher than that of the control group. Whereas, in the group administered with WFV in advance, a decrease in the weight ratio has a positive correlation with an increase in the dose. In the group of mice administered with 1000 mg/kg of WFV, the value level was significantly different from that of the cisplatin group, and had no significant difference compared with Amifostine serving as a nephroprotectant to cisplatin. See Table 1 below for each result.

TABLE 1

| | Dose | Body weight(g) | | Kidney | Kidney |
|---|---|---|---|---|---|
| Group | (mg/kg) | Before | After | weight(g) | index(mg/g) |
| N | — | 35.2 ± 0.61 | 39.6 ± 0.48 | 0.53 ± 0.029 | 1.34 ± 0.08 |
| Cis | — | 35.2 ± 0.35 | 32.5 ± 0.26 | 0.76 ± 0.034 | 2.35 ± 0.10^{###} |
| Cis + WFV(L) | 250 | 35.4 ± 0.21 | 34.9 ± 0.27 | 0.68 ± 0.012 | 1.94 ± 0.04*** |
| Cis + WFV(M) | 500 | 35.2 ± 0.28 | 35.5 ± 0.21 | 0.67 ± 0.022 | 1.88 ± 0.05*** |
| Cis + WFV(H) | 1000 | 35.2 ± 0.37 | 36.2 ± 0.39 | 0.61 ± 0.122 | 1.68 ± 0.03*** |
| Ami | 200 | 35.3 ± 0.27 | 36.6 ± 0.18 | 0.55 ± 0.025 | 1.50 ± 0.06*** |

### Example 2

Effect of cisplatin on renal function of mouse and protective properties of brown Flammulina velutipes water extract

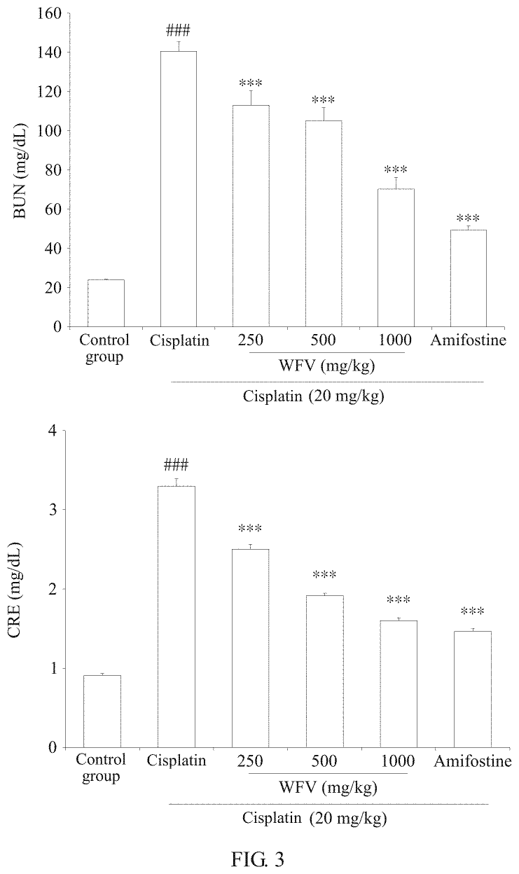

Referring to FIG. 3, FIG. 3 is a schematic view showing the effect of the control group, the cisplatin induction group, the positive control group, and the low-middle-high dose brown Flammulina velutipes water extract group on a kidney function of the mouse. As shown in FIG. 3, in the group of mice administered with cisplatin, the renal glomerular filtration rate was decreased, thereby causing an increase in blood urea nitrogen (BUN) and creatinine (CRE). This indicates that the kidney of the mouse was affected by the nephrotoxic side effect of cisplatin. Whereas, when brown Flammulina velutipes water extract (WFV) was provided in advance, the corresponding blood urea nitrogen and creatinine were decreased as the WFV dose was increased, and this result was similar to that of the Amifostine group.

### Example 3

Correlation between Flammulina velutipes water extract with inflammatory factors in mouse model of acute renal failure

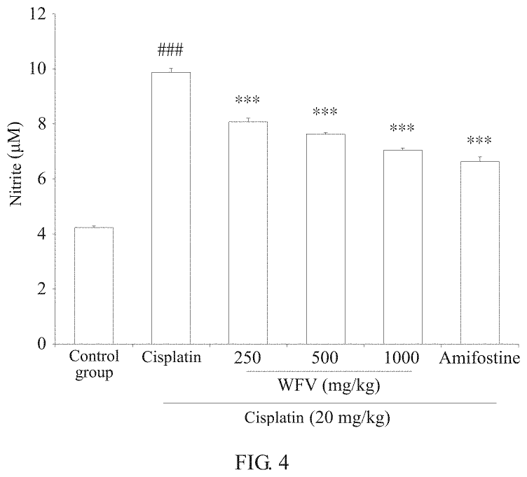

Referring to FIG. 4, FIG. 4 is a schematic view showing the effect of the control group, the cisplatin induction group, the positive control group, and the low-middle-high dose brown Flammulina velutipes water extract group on an NO free radicals in mouse serum. As shown in FIG. 4, under cisplatin induction, nitrite in mouse serum, that is, the expression level of NO free radicals was increased remarkably. This indicates the inflammatory response in the mouse was increased. Whereas, in the group administered with Flammulina velutipes water extract (WFV) in advance, it can be seen that the expression level of NO is decreased when the dose of WFV was increased, and the expression level of NO in the 1000 mg/kg group was close to that of the Amifostine group.

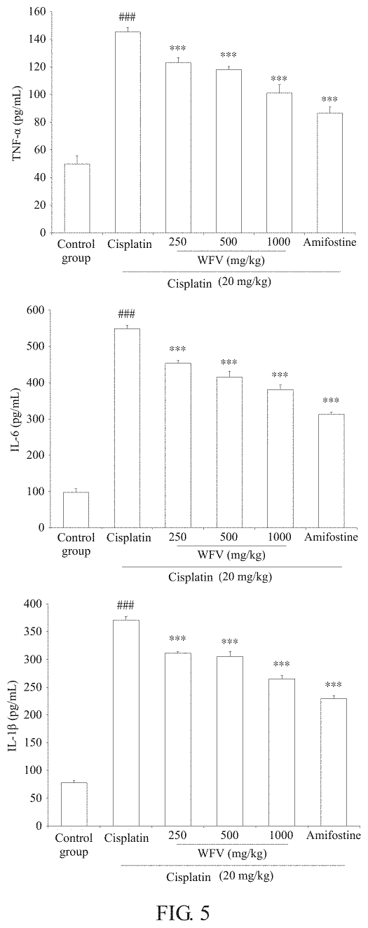

See FIG. 5 for the cytokine correlation measurement. FIG. 5 is a schematic view showing the effect of the control group, the cisplatin induction group, the positive control group, and the low-middle-high dose brown Flammulina velutipes water extract group on TNF-α, IL-1β and IL-6 of the mouse. As shown in FIG. 5, concentrations of the inflammatory factors of the three items TNF-α, IL-1β and IL-6 were increased in the cisplatin induction group compared with the control group. This indicates that the induction of cisplatin promotes the inflammatory response in the kidney. Whereas, when WFV was administered in advance, the concentration of the aforementioned inflammatory factors was decreased as the concentration of the used WFV was increased. In addition, the concentration gradually approached the Amifostine group as the positive control group.

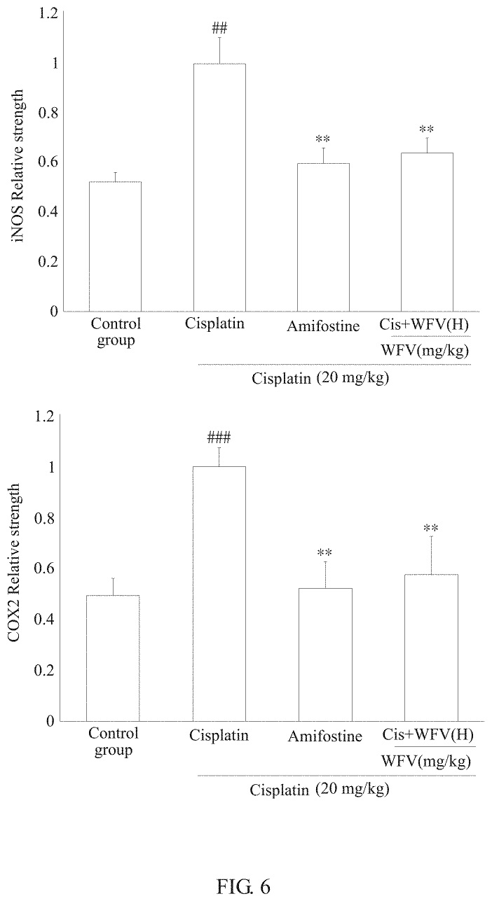

Meanwhile, referring to FIG. 6, FIG. 6 is a schematic view showing the effect of the control group, the cisplatin induction group, the positive control group, and the low-middle-high dose brown Flammulina velutipes water extract group on iNOS and COX-2 of the mouse. Regarding the expression levels of iNOS and COX-2 as inflammatory substances, the amount of protein in the present study was determined using Western blotting. According to the results, the inflammatory response in the kidney was promoted by cisplatin in the cisplatin induction group, and both iNOS and COX-2 expressed significant differences compared with the control group. Whereas, after Amifostine or high-dose WFV was administered in advance, both expression levels of iNOS and COX-2 were decreased.

### Example 4

Correlation between Flammulina velutipes water extracts and TLR4/NFκB in mouse model of acute kidney injury.

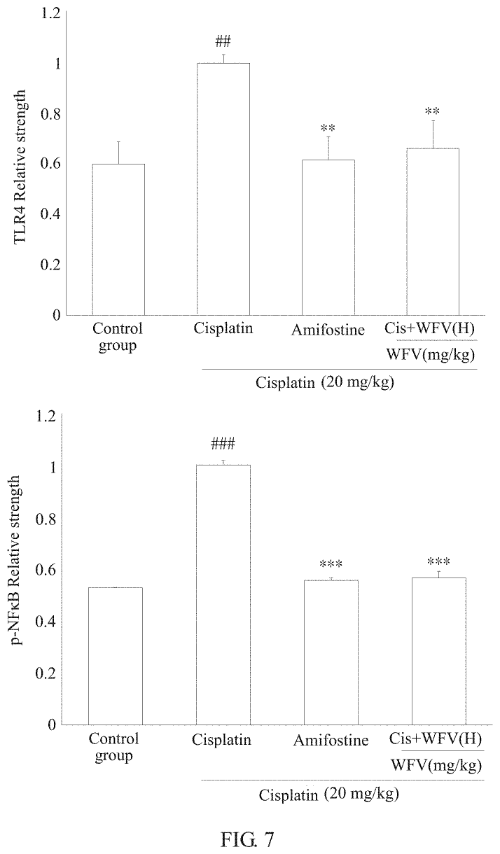

Referring to FIG. 7, FIG. 7 is a schematic view showing the effect of the control group, the cisplatin induction group, the positive control group, and the low-middle-high dose brown Flammulina velutipes water extract group on TLR4/NFκB of the mouse. As shown in FIG. 7, the cisplatin-induced renal inflammatory response in the mouse was increased. The expression levels of TLR4 and p-NFκB were further compared with the control group, and exhibited an upward trend. Whereas, after Amifostine or high-dose WFV was administered in advance, both expression levels of TLR4 and p-NFκB were decreased, and had a similar trend compared with the control group.

### Example 5

Effect of cisplatin on renal cell oxidative stress of mouse and protective properties of brown Flammulina velutipes water extract

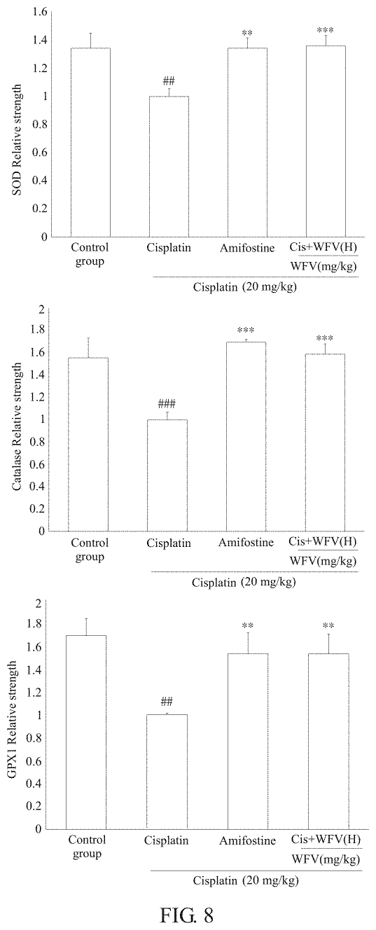

Referring to FIG. 8, FIG. 8 is a schematic view showing the effect of the control group, the cisplatin induction group, the positive control group, and the low-middle-high dose brown Flammulina velutipes water extract group on SOD, catalase and GPX1 of the mouse. As shown in FIG. 8, in terms of superoxide dismutase (SOD), catalase and glutathione peroxidase (GPX1), expression levels of SOD, catalase and GPX1 were decreased in mouse kidneys under cisplatin induction compared to the control group. In comparison, in the group administered with Amifostine and 1000 mg/kg of Flammulina velutipes water extract (WFV) in advance, the expression levels of SOD, catalase and GPX1 were not decreased. Whereas, a trend similar to the control group was exhibited. This appears to be protected from the effects of cisplatin on oxidative stress.

### Example 6

Correlation between Flammulina velutipes water extracts with Nrf2/HO-1 pathways in mouse model of acute renal failure

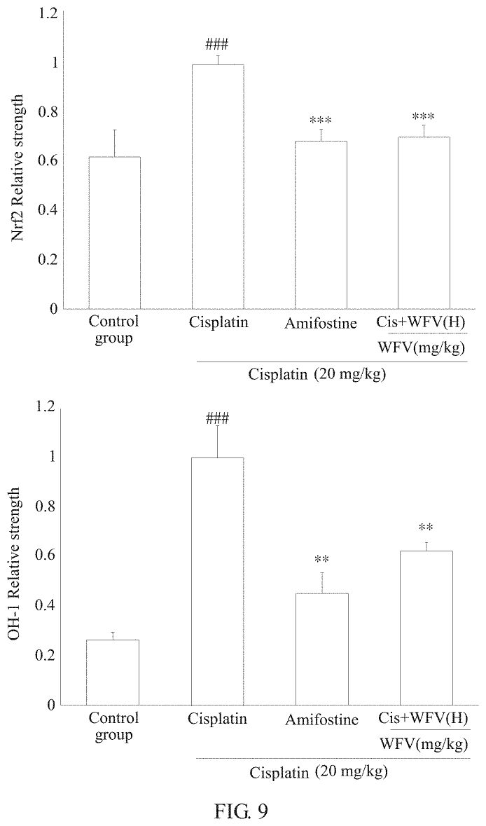

Referring to FIG. 9, FIG. 9 is a schematic view showing the effect of the control group, the cisplatin induction group, the positive control group, and the low-middle-high dose brown Flammulina velutipes water extract group on the expression level of Nrf2/HO-1 of the mouse. The expression of nuclear factor E2-related factor 2 (Nrf2)/hemosiderin oxidase-1 (HO-1) is associated with cell protection against oxidative stress. As shown in FIG. 9, in this experiment, the expression levels of Nrf2 and downstream HO-1 in the induction group were significantly increased due to the induction of cisplatin, thereby responding to oxidative stress applied to cells. Whereas, in the positive control group or the high-dose Flammulina velutipes water extract (WFV) group, the expression level was decreased and close to the control group. This indicates that administration before treatment is helpful in protection from cisplatin-induced oxidative stress.

### Example 7

Correlation between Flammulina velutipes water extracts and apoptosis pathway in mouse model of acute kidney injury

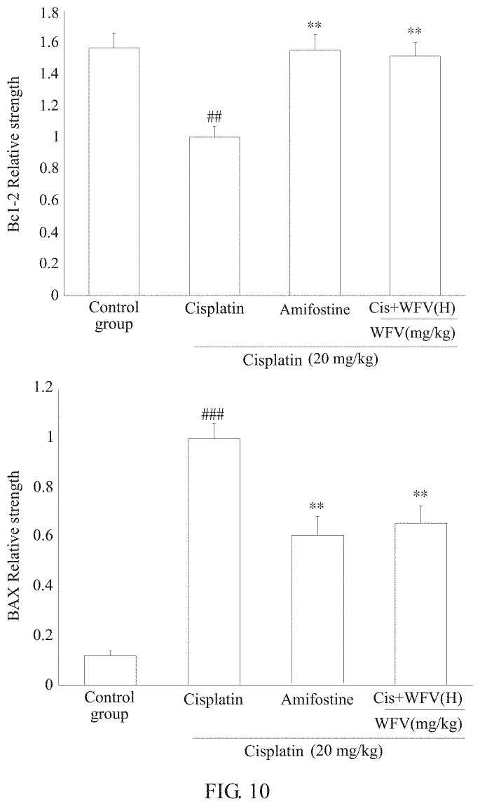

Referring to FIG. 10, FIG. 10 is a schematic view showing the effect of the control group, the cisplatin induction group, the positive control group, and the low-middle-high dose brown Flammulina velutipes water extract group on Bcl-2 and Bax of the mouse. For the pathway associated with apoptosis, in this study, Bcl-2 and Bax were selected as observation indexes, and Western blotting method was used to determine the difference in protein amount. As shown in FIG. 10, the expression level of B lymphocytoma-2 gene (Bcl-2) having a cytoprotective function was decreased in the cisplatin induction group, and significantly different from the control group. Whereas, in the Amifostine positive control group or the high-dose Flammulina velutipes water extract (WFV) group, the expression level of Bcl-2 was increased in both of the groups, and significantly different from the induction group. The expression level of apoptogenic protein BAX was increased in the cisplatin induction group, and significantly different from the control group. Whereas, after Amifostine or high-dose WFV was administered in advance, the expression level was significantly decreased.

### Example 8

Correlation between Flammulina velutipes water extracts and autophagy pathways in mouse model of acute kidney injury

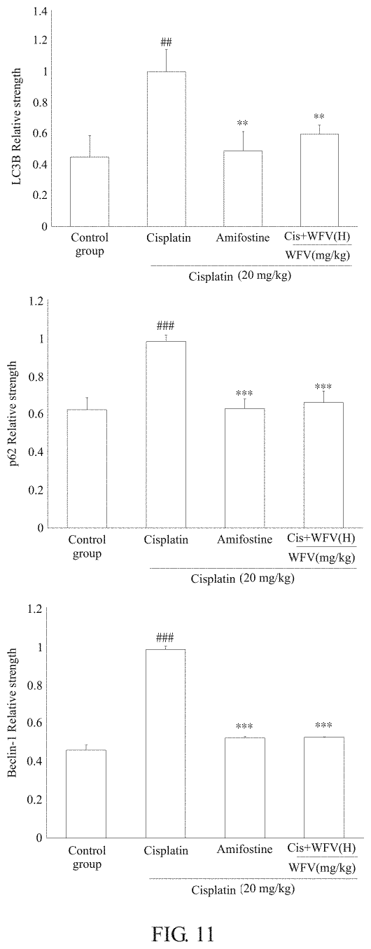

Referring to FIG. 11, FIG. 11 is a schematic view showing the effect of the control group, the cisplatin induction group, the positive control group, and the low-middle-high dose brown Flammulina velutipes water extract group on LC3B, p62 and Beclin-1 of the mouse. As shown in FIG. 11, with respect to the expressions of autophagy-related proteins LC3B, p62 and Beclin-1 compared with the control group, the levels of LC3B, p62 and Beclin-1 were significantly increased in mice administered with cisplatin. This indicates that renal cell damage due to cisplatin induction causes autophagy, thereby enabling the cells to survive. Whereas, in the mice of the positive control group and the group administered with high-dose Flammulina velutipes water extract (WFV) in advance, the expression level of the protein was reversed, similarly to the control group. This indicates that WFV protects mice from cisplatin-induced kidney injury.

### Example 9

Correlation between Flammulina velutipes water extract and PI3K/AKT pathways in mouse model of acute renal failure

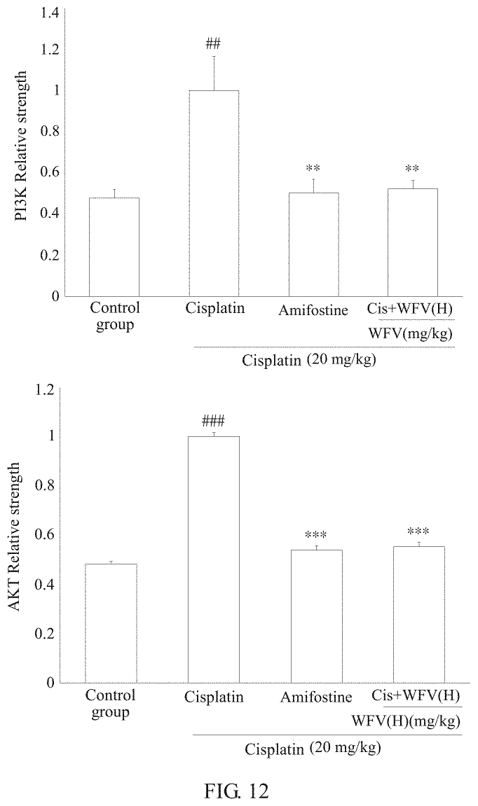

Referring to FIG. 12, FIG. 12 is a schematic view showing the effect of the control group, the cisplatin induction group, the positive control group, and the low-middle-high dose brown Flammulina velutipes water extract group on PI3K/AKT pathways of the mouse. Increased oxidative stress by cisplatin affects the PI3K/AKT pathways. In this experiment, phosphatidylinositol 3-kinase (PI3K)/protein kinase B (AKT) pathways were additionally studied using Western blotting method. As shown in FIG. 12, compared to the control group, the expression levels of PI3K and AKT were significantly increased in mice administered with cisplatin. Whereas, according to mice of the group administered with high-dose Flammulina velutipes water extracts (WFV) and the Amifostine positive control group, the protein expression levels were similar to that of the control group. This indicates that WFV can protect mice from cisplatin-induced high oxidative stress.

In conclusion, as can be seen from the examples, brown Flammulina velutipes water extract has the effect of significantly inhibiting cisplatin-induced acute kidney injury. For example, decrease in body weight and increase in kidney weight due to the kidney damage, increase in blood urea nitrogen and creatinine due to decrease in glomerular filtration rate, and increase in expression levels of various inflammatory factors can be reduced, and the protein expression level associated with acute renal failure can be can reduced. In addition, the expression levels of enzymes such as superoxide dismutase (SOD), catalase and glutathione peroxidase (GPX1) may be increased, so that the antioxidant capacity of kidney cells can be enhanced.

Those having ordinary skill in the art will understand from the aforementioned descriptions that the present invention can be exemplified in other specific forms without departing from the disclosed technical idea or essential features. Therefore, the exemplary aspects disclosed herein are for illustrative purposes only and will not be construed as limiting the scope of the present disclosure. The present disclosure may encompass various alterations, modifications, equivalents, and other aspects as defined by the following appended claims and may be included within the spirit and scope of the present disclosure in addition to the above exemplary aspects.

What is claimed is:

1. A use of a brown Flammulina velutipes extract for inhibiting an acute kidney injury caused by cisplatin, wherein the brown Flammulina velutipes extract includes water extract obtained by extraction from brown Flammulina velutipes using water as a solvent.

2. The use of claim 1, wherein the brown Flammulina velutipes extract reduces weight loss caused by cisplatin-induced acute kidney injury.

3. The use of claim 1, wherein the brown Flammulina velutipes extract reduces an increase in kidney weight due to a decrease in a glomerular filtration rate caused by cisplatin-induced acute kidney injury.

4. The use of claim 1, wherein the brown Flammulina velutipes extract reduces an increase in blood urea nitrogen and creatinine caused by cisplatin-induced acute kidney injury.

5. The use of claim 1, wherein the brown Flammulina velutipes extract reduces an amount of increased expression of an inflammatory factor caused by cisplatin-induced acute kidney injury, and
the inflammatory factor includes one or more selected from the group consisting of NO free radicals, TNF-α, IL-1β, IL-6, iNOS and COX-2.

6. The use of claim 1, wherein the brown Flammulina velutipes extract reduces amounts of increased expression of TLR4 and p-NFκB caused by cisplatin-induced acute kidney injury.

7. The use of claim 1, wherein the brown Flammulina velutipes extract alleviates a decrease in expression of antioxidant enzyme caused by cisplatin-induced acute kidney injury, and
the antioxidant enzyme includes one or more selected from the group consisting of superoxide dismutase, catalase and glutathione peroxidase (GPX1).

8. The use of claim 1, wherein the brown Flammulina velutipes extract alleviates amounts of increased expression of nuclear factor E2-related factor 2 (Nrf2), hemosiderin oxidase-1 (HO-1), apoptogenic protein BAX and/or autophagy-related proteins LC3B, p62 and Beclin caused by cisplatin-induced acute kidney injury.

9. The use of claim 1, wherein the brown Flammulina velutipes extract alleviates an amount of decreased expression of B lymphocytoma-2 gene (Bcl-2) caused by cisplatin-induced acute kidney injury.

10. The use of claim 1, wherein the brown Flammulina velutipes extract alleviates amounts of increased expression of phosphatidylinositol 3-kinase (PI3K) and protein kinase B (AKT) caused by cisplatin-induced acute kidney injury.
